(19) United States
(12) Patent Application Publication
(10) Pub. No.: US 2013/0202666 A1
(43) Pub. Date: Aug. 8, 2013

(54) HAIR TREATMENT COMPOSITION

(71) Applicants: Jordan Todorov Petkov, Bebington (GB); Glyn Roberts, Bebington (GB)

(72) Inventors: Jordan Todorov Petkov, Bebington (GB); Glyn Roberts, Bebington (GB)

(21) Appl. No.: 13/816,742

(22) PCT Filed: Aug. 16, 2011
(86) PCT No.: PCT/EP2011/064105

§ 371 Date: Apr. 9, 2013

(30) Foreign Application Priority Data
Aug. 20, 2010 (EP) 10173522.3

(51) Int. Cl. A61K 8/64; A61Q 5/00; A61Q 13/00

(52) U.S. Cl. CPC A61K 8/64; A61Q 13/00; A61Q 5/00
USPC 424/401; 424/70.14

(57) ABSTRACT

An aerated composition comprising, surfactant aggregates, perfume and a hydrophobin.

### TECHNICAL FIELD

This invention relates to aerated perfumed compositions.

### BACKGROUND

Perfume is a characteristic of many products that is important to consumers. Many perfumed products do not have the strength of perfume required without using a large quantity of perfume in the product.

US2009136433 (BASF) discloses what appear to be prophetic compositions comprising cationic surfactant and hydrophobin. They are not aerated. The hydrophobins are Class I fusion proteins and are said to be introduced into the compositions in order to deposit onto keratin.

The object of the present invention is to provide a composition that delivers an initial noticeable release of perfume or an impactful perfume.

### DESCRIPTION OF THE INVENTION

The present invention relates to a stable aerated composition comprising surfactant aggregates, perfume, hydrophobin at least 5% by volume of air at 20° C.

The invention also relates to a method of treating hair comprising the step of applying to the hair the composition described above.

### DETAILED DESCRIPTION OF THE INVENTION

The compositions of the invention deliver a powerful release of perfume from a product. Such powerful release of perfume is due to an increase in the perfume head space of the product.

The compositions of the agents of the invention are foamed with air or an inert gas up to a degree of foam-up which typically is at least 5 percent of air at 20° C., preferably 10 percent and up to 500 percent, preferably between 20 and 200 percent and particularly between 30 and 100 percent by volume. It is preferred that at least 40% volume of the product is air, more preferably 50% by volume.

The level of aeration can be measured using standard density measurements.

In the context of the present invention the definition of a stable foam is a product characterized in that it has is homogeneously distributed a gaseous substance in the form of small gas bubbles which remain in this homogeneous distribution over a period of at least one week, preferably at least one month and particularly at least 6 months if stored at room temperature 20° C.

Preferably, the average bubble size on initial manufacture is from 5 microns in diameter to 100 microns, preferably from 6 microns to 50 microns. It is preferable that the average bubble size is no more than 50 times its initial diameter, preferably no more than 40 times its original diameter after storage at 45° C. for 28 days. Preferably, the bubble size after 4 months storage at 45° is 500 microns or less, more preferably 300 microns or less.

Bubble size is based on the number average diameter.

Bubble size diameters are measured using an Olympus microscope, camera and associated AnalySIS software.

### Hydrophobin

The composition of the invention comprises at least one hydrophobin.

Hydrophobins are a well-defined class of proteins (Wessels, 1997, Adv. Microb. Physio. 38: 1-45; Wosten, 2001, Annu Rev. Microbiol. 55: 625-646) capable of self-assembly at a hydrophobic/hydrophilic interface, and having a conserved sequence:

(SEQ ID No. 1)
Xₙ-C-X₅₋₉-C-C-X₁₁₋₃₉-C-X₈₋₂₃-C-X₅₋₉-C-C-X₆₋₁₈-C-
Xₘ

where X represents any amino acid, and n and m independently represent an integer. Typically, a hydrophobin has a length of up to 125 amino acids. The cysteine residues (C) in the conserved sequence are part of disulphide bridges. In the context of this invention, the term hydrophobin has a wider meaning to include functionally equivalent proteins still displaying the characteristic of self-assembly at a hydrophobic-hydrophilic interface resulting in a protein film, such as proteins comprising the sequence:

(SEQ ID No. 2)
Xₙ-C-X₁₋₅₀-C-X₀₋₅-C-X₁₋₁₀₀-C-X₁₋₁₀₀-C-X₁₋₅₀-C-
X₀₋₅-C-X₁₋₅₀-C-Xₘ

or parts thereof still displaying the characteristic of self-assembly at a hydrophobic-hydrophilic interface resulting in a protein film. In accordance with the definition of this invention, self-assembly can be detected by adsorbing the protein to Teflon and using Circular Dichroism to establish the presence of a secondary structure (in general, α-helix) (De Vocht et al., 1998, Biophys. J. 74: 2059-68).

The formation of a film can be established by incubating a Teflon sheet in the protein solution followed by at least three washes with water or buffer (Wosten et al., 1994, Embo. J. 13: 5848-54). The protein film can be visualised by any suitable method, such as labelling with a fluorescent marker or by the use of fluorescent antibodies, as is well established in the art. m and n typically have values ranging from 0 to 2000, but more usually m and n in total are less than 100 or 200. The definition of hydrophobin in the context of this invention includes fusion proteins of a hydrophobin and another polypeptide as well as conjugates of hydrophobin and other molecules such as polysaccharides.

Hydrophobins identified to date are generally classed as either class I or class II. Both types have been identified in fungi as secreted proteins that self-assemble at hydrophobic-hydrophilic interfaces into amphipathic films.

Hydrophobin-like proteins have also been identified in filamentous bacteria, such as Actinomycete and Streptomyces sp. (WO01/74864; Talbot, 2003, Curr. Biol, 13: R696-R698). These bacterial proteins by contrast to fungal hydrophobins, may form only up to one disulphide bridge since they may have only two cysteine residues. Such proteins are an example of functional equivalents to hydrophobins having the consensus sequences shown in SEQ ID Nos. 1 and 2, and are within the scope of this invention.

The hydrophobins can be obtained by extraction from native sources, such as filamentous fungi, by any suitable process. For example, hydrophobins can be obtained by culturing filamentous fungi that secrete the hydrophobin into the growth medium or by extraction from fungal mycelia with 60% ethanol. It is particularly preferred to isolate hydrophobins from host organisms that naturally secrete hydrophobins. Preferred hosts are hyphomycetes (e.g. Trichoderma), basidiomycetes and ascomycetes. Particularly preferred hosts are food grade organisms, such as Cryphonectria parasitica which secretes a hydrophobin termed cryparin (MacCabe and Van Alfen, 1999, App. Environ. Microbiol 65: 5431-5435).

Alternatively, hydrophobins can be obtained by the use of recombinant technology. For example host cells, typically micro-organisms, may be modified to express hydrophobins and the hydrophobins can then be isolated and used in accordance with the present invention. Techniques for introducing nucleic acid constructs encoding hydrophobins into host cells are well known in the art. More than 34 genes coding for hydrophobins have been cloned, from over 16 fungal species (see for example WO96/41882 which gives the sequence of hydrophobins identified in Agaricus bisporus; and Wosten, 2001, Annu. Rev. Microbiol. 55: 625-646). Recombinant technology can also be used to modify hydrophobin sequences or synthesise novel hydrophobins having desired/improved properties.

Typically, an appropriate host cell or organism is transformed by a nucleic acid construct that encodes the desired hydrophobin. The nucleotide sequence coding for the polypeptide can be inserted into a suitable expression vector encoding the necessary elements for transcription and translation and in such a manner that they will be expressed under appropriate conditions (e.g. in proper orientation and correct reading frame and with appropriate targeting and expression sequences). The methods required to construct these expression vectors are well known to those skilled in the art.

A number of expression systems may be used to express the polypeptide coding sequence. These include, but are not limited to, bacteria, fungi (including yeast), insect cell systems, plant cell culture systems and plants all transformed with the appropriate expression vectors. Preferred hosts are those that are considered food grade—‘generally regarded as safe’ (GRAS).

Suitable fungal species, include yeasts such as (but not limited to) those of the genera Saccharomyces, Kluyveromyces, Pichia, Hansenula, Candida, Schizo saccharomyces and the like, and filamentous species such as (but not limited to) those of the genera Aspergillus, Trichoderma, Mucor, Neurospora, Fusarium and the like.

The sequences encoding the hydrophobins are preferably at least 80% identical at the amino acid level to a hydrophobin identified in nature, more preferably at least 95% or 100% identical. However, persons skilled in the art may make conservative substitutions or other amino acid changes that do not reduce the biological activity of the hydrophobin. For the purpose of the invention these hydrophobins possessing this high level of identity to a hydrophobin that naturally occurs are also embraced within the term “hydrophobins”.

Hydrophobins can be purified from culture media or cellular extracts by, for example, the procedure described in WO01/57076 which involves adsorbing the hydrophobin present in a hydrophobin-containing solution to surface and then contacting the surface with a surfactant, such as Tween 20, to elute the hydrophobin from the surface. See also Collen et al., 2002, Biochim Biophys Acta. 1569: 139-50; Calonje et al., 2002, Can. J. Microbiol. 48: 1030-4; Askolin et al., 2001, Appl Microbiol Biotechnol. 57: 124-30; and De Vries et al., 1999, Eur J Biochem. 262: 377-85.

Typically, the hydrophobin is in an isolated form, typically at least partially purified, such as at least 10% pure, based on weight of solids. By “isolated form”, we mean that the hydrophobin is not added as part of a naturally-occurring organism, such as a mushroom, which naturally expresses hydrophobins. Instead, the hydrophobin will typically either have been extracted from a naturally-occurring source or obtained by recombinant expression in a host organism.

Hydrophobin proteins can be divided into two classes: Class I, which are largely insoluble in water, and Class II, which are readily soluble in water.

Preferably, the hydrophobins chosen are Class II hydrophobins. More preferably the hydrophobins used are Class II hydrophobins such as HFBI, HFBII, HFBIII.

Most preferably it is HFBII.

The hydrophobin can be from a single source or a plurality of sources e.g. a mixture of two or more different hydrophobins.

The total amount of hydrophobin in compositions of the invention will generally be at least 0.001%, more preferably at least 0.005 or 0.01%, and generally no greater than 2% by total weight hydrophobin based on the total weight of the composition.

The hydrophobin surprisingly provides a stable foam of the cationic surfactant composition, even at the higher temperatures where bubbles would normally be expected to coalesce or escape from the composition. Furthermore, this is achieved without compromising the ease of spread and ease of rinse of the composition.

### Perfume

Compositions of the invention comprise a perfume, preferably the level of perfume is from 0.01 to 2 wt %, more preferably from 0.1 to 1 wt %.

### Cationic Surfactant

Suitable cationic surfactants can be used singly or in admixture. Preferably the cationic surfactant is a cationic conditioning surfactant.

Preferably, the cationic conditioning surfactant is a quaternary ammonium or an amine having at least one long chain alkyl group has had on average around about 16 to about 40 carbon atoms.

Suitable cationic surfactants for use include cetyltrimethylammonium chloride, behenyltrimethylammonium chloride, cetylpyridinium chloride, tetramethylammonium chloride, tetraethylammonium chloride, octyltrimethylammonium chloride, dodecyltrimethylammonium chloride, hexadecyltrimethylammonium chloride, octyldimethylbenzylammonium chloride, decyldimethylbenzylammonium chloride, stearyldimethylbenzylammonium chloride, didodecyldimethylammonium chloride, dioctadecyldimethylammonium chloride, tallowtrimethylammonium chloride, cocotrimethylammonium chloride, and the corresponding hydroxides thereof. Further suitable cationic surfactants include those materials having the CTFA designations Quaternium-5, Quaternium-31 and Quaternium-18. Mixtures of any of the foregoing materials may also be suitable.

Preferably, the cationic surfactant is insoluble. Insoluble in this context is defined as materials which at 20° C. do not form isotropic, clear solutions in water at greater than 0.2 Wt %.

Preferred cationic surfactants are moncationic, more preferred surfactants include the compounds distearyldimethylammonium, dicetyldimethylammonium, tricetylmethylammonium,-behenyltrimethylammonium, stearyl benzyl dimethylammonium, suitable amines include distearylamine, distearylmethylamine, behenylamine, behenylmethylamine, behenyldimethylamine, dicetylamine, dicetylmethylamine, tricetylamine.

Preferably, the cationic salt is a combination of behenyltrimethylammonium/salt with a second cationic conditioning surfactant. In the most preferred form the cationic conditioning surfactant is behenyltrimethylammonium salt, in particular the chloride.

In compositions of the invention, the level of cationic surfactant is preferably from 0.1 to 10%, more preferably 0.5 to 7%, most preferably 1 to 5% by weight of the total composition.

### Fatty Alcohol

Compositions of the invention advantageously incorporate a fatty alcohol material. The combined use of fatty alcohol materials and cationic surfactants in compositions is believed to be especially advantageous, because this leads to the formation of a lamellar phase, in which the cationic surfactant is dispersed.

Representative fatty alcohols comprise from 8 to 22 carbon atoms, more preferably 16 to 20. Examples of suitable fatty alcohols include cetyl alcohol, stearyl alcohol and mixtures thereof. The use of these materials is also advantageous in that they contribute to the overall conditioning properties of compositions of the invention.

The level of fatty alcohol material in compositions of the invention is conveniently from 0.01 to 10%, preferably from 0.1 to 5% by weight of the composition. The weight ratio of cationic surfactant to fatty alcohol is suitably from 10:1 to 1:10, preferably from 4:1 to 1:8, optimally from 1:1 to 1:4.

### Further Ingredients

Other ingredients may include viscosity modifiers, preservatives, silicones, colouring agents, polyols such as glycerine and polypropylene glycol, chelating agents such as EDTA, antioxidants such as vitamin E acetate, fragrances, antimicrobials and sunscreens. Each of these ingredients will be present in an amount effective to accomplish its purpose. Generally these optional ingredients are included individually at a level of up to about 5% by weight of the total composition.

Preferably, compositions of this invention also contain adjuvants suitable for personal, more preferably hair care. Generally such ingredients are included individually at a level of up to 2%, preferably up to 1%, by weight of the total composition.

Among suitable adjuvants, are:
(i) natural hair root nutrients, such as amino acids and sugars. Examples of suitable amino acids include arginine, cysteine, glutamine, glutamic acid, isoleucine, leucine, methionine, serine and valine, and/or precursors and derivatives thereof. The amino acids may be added singly, in mixtures, or in the form of peptides, e.g. di- and tripeptides. The amino acids may also be added in the form of a protein hydrolysate, such as a keratin or collagen hydrolysate. Suitable sugars are glucose, dextrose and fructose. These may be added singly or in the form of, e.g. fruit extracts.
(ii) hair fibre benefit agents. Examples are:
   ceramides, for moisturising the fibre and maintaining cuticle integrity. Ceramides are available by extraction from natural sources, or as synthetic ceramides and pseudoceramides. A preferred ceramide is Ceramide II, ex Quest. Mixtures of ceramides may also be suitable, such as Ceramides LS, ex Laboratoires Serobiologiques.
   free fatty acids, for cuticle repair and damage prevention. Examples are branched chain fatty acids such as 18-methyleicosanoic acid and other homologues of this series, straight chain fatty acids such as stearic, myristic and palmitic acids, and unsaturated fatty acids such as oleic acid, linoleic acid, linolenic acid and arachidonic acid. A preferred fatty acid is oleic acid. The fatty acids may be added singly, as mixtures, or in the form of blends derived from extracts of, e.g. lanolin.

Mixtures of any of the above active ingredients may also be used.

### Structure of Composition

It is preferred if the structure of the composition is that of an aggregate, preferably the composition has a lamellar structure. It is preferred if the composition does not have a micellar structure.

### Mode of Use

The compositions of the invention are primarily intended for topical application to the body, preferably the hair and/or scalp of a human subject in rinse-off or leave-on compositions.

The compositions provided by the invention may be aqueous conditioner compositions, used by massaging them into the hair followed by rinsing with clean water prior to drying the hair.

The invention will be further described by way of the following non-limiting examples.

### EXAMPLES

A hair conditioner composition was made as specified in Table 1 using the following preparative method.

TABLE 1
Composition 1

| | Trade | % | Composition | | |
|---|---|---|---|---|---|
| Chemical Name | name | Active | 1 | A | B |
| Methyl-p-hydroxy | Nipagin M | 100.00 | 0.200 | 0.200 | 0.200 |
| benzoate | | | | | |
| BTAC | Genamin | 70.00 | 2.850 | 2.850 | 2.850 |
| | BTLF | | | | |
| Stearyl Alcohol | Lanette S3 | 100.00 | 4.000 | 4.000 | 4.000 |
| Perfume | perfume | 100.00 | 0.600 | 0.600 | 0.600 |
| Hydrophobin II | VTT HFB II | 0.92 | 0.1 | | |
| Water | | | To | To | To 100.00 |
| | | | 100.00 | 100.00 | |
| | | | Aerated | Aerated | Not |
| | | | | | Aerated |

1. Add the water and start the mixer at 60 rpm (˜40%)
2. Start to heat to 85° C. and add Nipagin M
3. At approx 80° C. (˜25 min) add Gemamin BTLF
4. At 85° C. (˜5 min) add Lanette S3 slowly OTT
5. Seal the mixer and turn on the vacuum until the bubble start to rise
6. Turn on the homogeniser at ˜40% and mix for 10 min
7. Leave to stir for a further 10 min
8. Cool the jacket to 65° C. and add quench water through the hopper
9. Cool to 30° C. and add perfume through the viewing port and stir for 5 min
10. Turn on vacuum until the bubbles rise and homogenise at 40% for 5 min
11. Stir for a further 5 to 10 min then discharge
12. The Hydrophobin was then added to 85 g of this base and made up to 100 g with water.
13. Aeration was achieved by aeration with a Bamix ‘Gordon Ramsey’ 200 W processor; a lid was used to prevent loss of perfume. The processor was run for 60 seconds. The level of aeration was 50% by volume.

Hydrophobin HFBII was obtained from VTT Biotechnology, Finland. It had been purified from Trichoderma reesei essentially as described in WO00/58342 and Linder et al., Biomacromolecules 2: 511-517.

1 g of each sample was transferred to identical glass vials, crimped and sealed. The compositions above then were stored at 25° C. for 2 weeks. The headspace above the composition was analysed by GC/MS and peak heights reported.

The results are shown in the following tables.

TABLE 2

| Retention time of | Average Peak Height | | |
|---|---|---|---|
| Headspace Component | (Mean of n = 3 samples) | | |
| (mins) | Composition 1 | Composition A | Composition B |
| 5.40 | 1740089.0 | 1480032.3 | 1383751.3 |
| 5.90 | 3464002.5 | 3090218.0 | 2600762.7 |
| 6.09 | 4526095.5 | 4232199.0 | 3983165.0 |
| 7.09 | 6813504.7 | 6560610.7 | 5962553.0 |
| 7.61 | 5242113.0 | 3530127.7 | 3155587.7 |
| 8.44 | 6159021.5 | 4283343.7 | 3602690.3 |
| 8.87 | 3818688.0 | 3876171.0 | 3327390.3 |
| 8.97 | 15074140.7 | 13575462.3 | 11712812.3 |
| 10.17 | 3399144.5 | 1705080.7 | 1384329.3 |
| 11.52 | 26304702.7 | 13935686.7 | 8784571.7 |
| 11.88 | Not detected or | 2290765.3 | 1762567.7 |
| | beneath | | |
| | detection limit | | |
| 13.88 | 15300848.0 | 4212030.3 | 1715776.3 |

Table 2 demonstrates that the majority of perfume components have higher peaks for the Examples of the invention compared with the comparative Examples. Higher peaks measured by GC relate directly to an increase in perfume impact.

The following is a further Example of a composition according to the invention. The Example is made according to Example 1.

TABLE 3
Example 2

| Chemical Name | Trade name | % Active | % |
|---|---|---|---|
| Methyl-p-hydroxy benzoate | Nipagin M | 100.00 | 0.200 |
| BTAC | Genamin BTLF | 70.00 | 4.28 |
| Stearyl Alcohol | Lanette S3 | 100.00 | 6.00 |
| Perfume | Perfume | 100.00 | 0.60 |
| Hydrophobin II | VTT HFB II | 0.92 | 0.1 |
| Water | | | To 100.00 |

1. A stable aerated composition comprising a lamellar phase comprising cationic surfactant, perfume, hydrophobia and at least 5% by volume of air at 20° C.

2. (canceled)

3. An aerated composition according to claim 1 in which the surfactant aggregates further comprise a fatty alcohol.

4. An aerated composition according to claim 3 in which the ratio of cationic surfactant to fatty alcohol is from 1:1 to 1:4.

5. An aerated composition according to claim 1 in which at least 50% volume of the product comprises air.

6. An aerated composition according to claim 1 comprising bubbles, the bubbles having a initial average diameter size from 5 to 100 microns.

7. An aerated treatment composition according to claim 6 in which the average bubble size after 4 months storage at 45° C. is 300 microns or less.

8. An aerated composition according to claim 1 in which the cationic surfactant is insoluble in water at 20° C.

9. An aerated composition according to claim 1 in which the cationic surfactant comprises a derivative of quaternary ammonium or an amine having at least one long chain alkyl group has had on average around about 16 to about 40 carbon atoms.

10. An aerated composition according to claim 8 in which the cationic surfactant is behenyltrimethylammonium or salt thereof.

11. An aerated composition according to claim 1 in which the hydrophobin is a class II hydrophobin HFBII.

12. An aerated composition according to claim 1 that is a hair treatment composition.

13. A method of treating hair comprising the step of applying to the hair the aerated composition described in claim 1.
